(19) United States
(12) Patent Application Publication
(10) Pub. No.: US 2025/0270231 A1
(43) Pub. Date: Aug. 28, 2025

(54) ULOTARONT HYDROCHLORIDE MANUFACTURING PROCESS

(71) Applicant: Sumitomo Pharma America, Inc., Marlborough, MA (US)

(72) Inventors: Xiaoxia CHEN, Marlborough, MA (US); Shahla JAMZAD, Marlborough, MA (US); John SNOONIAN, Marlborough, MA (US); Noreen ZAMAN, Marlborough, MA (US); Haitao ZHANG, Marlborough, MA (US)

(73) Assignee: Sumitomo Pharma America, Inc., Marlborough, MA (US)

(21) Appl. No.: 19/059,437

(22) Filed: Feb. 21, 2025

Related U.S. Application Data
(60) Provisional application No. 63/557,163, filed on Feb. 23, 2024.

(51) Int. Cl. C07D 495/04; A61K 9/16; A61K 9/20; A61K 31/381

(52) U.S. Cl. CPC C07D 495/04; A61K 9/16; A61K 9/20; A61K 31/381

(57) ABSTRACT

This disclosure provides improved methods of manufacturing ulotaront HCl, for reducing impurities in ulotaront HCl, and for processing ulotaront HCl into finished dosage forms.

### CROSS REFERENCE TO RELATED APPLICATIONS

This application is a U.S. Non-Provisional Application which claims priority to U.S. Provisional Application No. 63/557,163 filed Feb. 23, 2024, the contents of which are hereby incorporated by reference herein in their entirety.

### FIELD

The present disclosure relates to ulotaront hydrochloride, and to improved methods, compositions, and intermediates for manufacturing and processing ulotaront hydrochloride into finished dosage forms.

### BACKGROUND

Ulotaront hydrochloride, or ulotaront HCl, is known chemically as (S)-1-(4,7-dihydro-5H-thieno[2,3-c]pyran-7-yl)-N-methylmethanamine hydrochloride. Its molecular formula is C₉H₁₃NOS ⋅HCl and its molecular weight is 219.73. The chemical structure is:

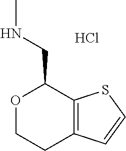

Ulotaront is sometimes referred to herein as Compound 1. In like manner, ulotaront hydrochloride is sometimes referred to herein as the HCl or hydrochloride salt of Compound 1.

Methods of manufacturing ulotaront (including ulotaront HCl) and processing ulotaront and ulotaront HCl into finished dosage forms are disclosed, inter alia, in US 2013/0109677 A1 to Shao et al. and US 2019/0256525 A1 to Bauer et al. Various impurities from the manufacturing process are described in paragraphs and of US 2019/0256525 A1.

Because ulotaront HCl is a pharmaceutical product intended for human administration, improvements in the manufacture of ulotaront HCl and finished dosage forms thereof are desirable to ensure the utmost consistency during manufacture, and purity and safety of the finished product.

### SUMMARY

In one embodiment, the disclosure provides a solid drug formulation comprising granulates comprising 30 to 120 mg of ulotaront HCl particles, in combination with one or more intra-granular excipients selected from a disintegrant, a diluent, a lubricant, or a combination thereof; wherein the ulotaront HCl particles have a D50 of from 37 to 98 μm.

In another embodiment, the disclosure provides ulotaront HCl particles having a D50 of from 37 to 98 μm.

In another embodiment, the disclosure provides a composition comprising ulotaront HCl and a compound selected from: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥ 0.001%; (c) Compound 4 in an amount of ≤0.15%, 0.08%, or 0.05% w/w, preferably ≥ 0.001%; or (d) Compound 5 in an amount of ≤0.60%, 0.30%, 0.10%, or 0.05% w/w, preferably ≥0.001%; wherein the percentages are based on the weight of the free base of ulotaront and the free base of the compound.

In another embodiment, the disclosure provides a solid drug formulation comprising granulates comprising 30 to 120 mg of ulotaront HCl particles in combination with one or more intra-granular excipients selected from a disintegrant, a diluent, a lubricant, or a combination thereof, further comprising a compound selected from: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥0.001%; (c) Compound 4 in an amount of ≤0.15%, 0.08%, or 0.05% w/w, preferably ≥0.001%; or (d) Compound 5 in an amount of ≤0.60%, 0.30%, 0.10%, or 0.05% w/w, preferably ≥0.001%; wherein the percentages are based on the weight of the free base of ulotaront and the free base of the compound.

In another embodiment, the disclosure provides ulotaront HCl comprising from 0.01% to 0.5% w/w water.

In another embodiment, the disclosure provides a solid drug formulation comprising 30 to 120 mg of ulotaront HCl comprising from 0.01% to 0.5% w/w water in combination with one or more excipients selected from a disintegrant, a diluent, a lubricant, or a combination thereof.

In another embodiment, the disclosure provides a method of making a pharmaceutical dosage form comprising: (a) providing a lot of ulotaront HCl comprising an amount of impurity, wherein the impurity is selected from Compound 2, Compound 3, Compound 4, Compound 5, or a salt thereof; (b) chromatographically separating the impurity from the ulotaront HCl in a sample from the lot and determining the amount of impurity in the sample; and (c) if the amount of the impurity in the sample is less than an acceptance threshold, processing the lot into one or more finished dosage forms.

In another embodiment, the disclosure provides a hemihydrate R-mandelate salt of Compound 3.

In another embodiment, the disclosure provides a method of making a hemihydrate R-mandelate salt of Compound 3 comprising crystallizing a R-mandelate salt of Compound 3 from a solution comprising acetone or acetonitrile and ≥0.7% water.

In another embodiment, the disclosure provides a method of making ulotaront HCl or a finished dosage form comprising ulotaront HCl comprising: (a) contacting a composition comprising a R-mandelate salt of ulotaront and a hemihydrate R-mandelate salt of Compound 3 with a base to form the free base of ulotaront, and (b) contacting the free base with anhydrous HCl to form ulotaront HCl.

In another embodiment, the disclosure provides a mixture comprising ulotaront HCl and Compound 2 or a salt thereof.

In another embodiment, the disclosure provides a mixture comprising ulotaront HCl and Compound 4 or a salt thereof.

In another embodiment, the disclosure provides a mixture comprising ulotaront HCl and Compound 5 or a salt thereof.

In another embodiment, the disclosure provides a hemihydrate R-mandelate salt of Compound 3 characterized by a powder x-ray diffraction pattern comprising three or more peaks, in terms of 2-theta, at 14.02±0.2°, 17.16±0.2°, 22.96±0.2°, 27.68±0.2°, 34.54±0.2°, 35.28±0.2°, and 35.7±0.2°.

In another embodiment the disclosure provides an anhydrous R-mandelate salt of Compound 3 characterized by a powder x-ray diffraction pattern comprising three or more peaks, in terms of 2-theta, at 22.24±0.2°, 23.4±0.2°, 23.9±0.2°, and 25.86±0.2°.

Additional advantages of the disclosure are set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate several embodiments of the disclosure and together with the description serve to explain the principles of the disclosure.

FIG. 1 depicts compression profiles for the 8 blends of Tablet Lot 1 under 2 stage FC mode (Target dwell time of 120 ms) and CS mode simulating Fette 3090 at 40 rpm press speed for flat tablets at 30% w/w API loading, as reported in Example 1. X/Y/Z in the legend indicates API Lot #, API D50, and Compression Mode.

FIG. 2 depicts compression profiles for the 8 blends of Tablet Lot 1 under 2 stage FC mode (Target dwell time of 120 ms) and CS mode simulating Fette 3090 at 40 rpm press speed for flat tablets at 40% w/w API loading, as reported in Example 1. X/Y/Z in the legend indicates API Lot #, API D50, and Compression Mode.

FIG. 3 depicts compression profiles for the 8 blends of Tablet Lot 1 under 2 stage FC mode (Target dwell time of 120 ms) and CS mode simulating Fette 3090 at 40 rpm press speed for biconvex tablets at 30% w/w API loading, as reported in Example 1. X/Y/Z in the legend indicates API Lot #, API D50, and Compression Mode.

FIG. 4 depicts compression profiles for the 8 blends of Tablet Lot 1 under 2 stage FC mode (Target dwell time of 120 ms) and CS mode simulating Fette 3090 at 40 rpm press speed for biconvex tablets at 40% w/w API loading, as reported in Example 1. X/Y/Z in the legend indicates API Lot #, API D50, and Compression Mode.

FIG. 5 is a graph depicting compaction profiles of batches made with the three milled ulotaront HCl lots with different particle size diameters PSD reported in Table 6. Mean and standard deviation (SD) are shown.

FIG. 6 is a graph depicting stratified content uniformity results for drug product batches from the drug substance particle size diameter (PSD) range study reported in Example 2.

FIG. 7 depicts XRD patterns of the anhydrous and hemihydrate R-mandelate salts of Compound 3 wherein, from the bottom up, the first line represents the anhydrous form; the second line represents the hemihydrate form; the third line represents a post DVS sample (hemihydrate form); the fourth line represents a lab sample of mixed forms.

FIG. 8 is a bar chart depicting the solubility of the anhydrous and hemihydrate R-mandelate salts of Compound 3 in the presence of the R-mandelate salt of Compound 1, where the forward slash bar depicts the solubility of the R-mandelate salt of anhydrous Compound 3 when combined with an equal amount of the R-mandelate salt of Compound 1, and the backward slash bar depicts the solubility of the R-mandelate salt of hemihydrate Compound 3 when combined with an equal amount of the R-mandelate salt of Compound 1.

FIG. 9 is a chart depicting the effect of in-process water concentrations on the amount of Compound 3 (mandelate salt) detected in an intermediate wet cake during various Compound 1 manufacturing campaigns.

### DETAILED DESCRIPTION

### Definitions

All published documents cited herein are hereby incorporated herein by reference in their entirety.

Reference in the specification to “one embodiment,” “an embodiment,” “one aspect,” or “an aspect” means that a particular feature, structure, or characteristic described in connection with the embodiment or aspect is included in at least one embodiment or aspect of the teachings. As used herein, the singular forms “a,” “an” and “the” are intended to include the plural forms as well, unless the context clearly indicates otherwise.

Unless otherwise specified, the word “includes” (or any variation thereon, e.g., “include,” “including,” etc.) is intended to be open-ended. For example, “A includes 1, 2 and 3” means that A includes, but is not limited to, 1, 2 and 3.

“Test methods”: When a physical property is recited herein, and a test method for that physical property is given in the examples hereto, it will be understood that the physical property is evaluated by those methods. When necessary, USP standards (as of Dec. 31, 2023) or, secondarily, ASTM standards (as of Dec. 31, 2023), can be used to supplement the test methods and, when no test methods are given, can be used as the test method. Coulometric titration (Karl Fischer) is applied for the determination of water content (USP <921>1c (Dec. 31, 2023)).

The term “ulotaront,” when standing by itself, refers to the free base form of (S)-1-(4,7-dihydro-5H-thieno[2,3-c]pyran-7-yl)-N-methylmethanamine. In like manner, Compound 2, 3, 4, 5, or 6, when standing by itself, refers to the free base form of Compound 2, 3, 4, 5, or 6.

The following Table A gives the free form of representative structures of compounds disclosed herein:

TABLE A

| | | | | | |
|---|---|---|---|---|---|
| 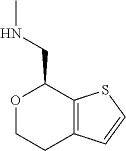 | 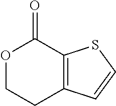 | 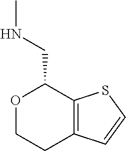 | 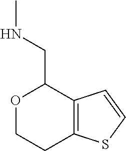 | 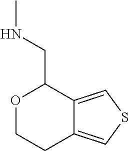 | 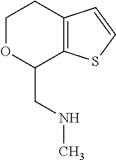 |
| Compound 1 | Compound 2 | Compound 3 | Compound 4 | Compound 5 | Compound 6* |

*Compound 6 is a racemic mixture of Compound 1 and Compound 3.

When an “acceptance threshold” is given as less than or equal to a particular percentage for any of the foregoing compounds, it will be understood that a manufacturer would practice the given threshold as long its specification for the compound was set at or below the recited percentage.

As used herein, the term “prominent peak,” in the context of an XRPD, means a peak with a greater than about 15% relative intensity. As used herein, the term “insignificant peak,” in the context of an XRPD, means a peak with a less than about 2% relative intensity. When characterizing the hemihydrate and anhydrate crystalline forms of the R-mandelate salt of Compound 3, one can select any three or more distinct prominent peaks from the chromatograms of the two crystalline forms to define a distinct crystalline form, where distinctness is judged by diffraction angles within +/−0.1 or +/−0.2 degrees.

As used herein, the term “polymorphic purity” refers to the weight % that is the specified polymorph form. For example, when a hemihydrate R-mandelate salt of Compound 3 is characterized as having greater than 95% polymorphic purity, it means that greater than 95% by weight of the substance is the hemihydrate R-mandelate salt of Compound 3 and less than 5% by weight of any other polymorph of the R-mandelate salt of Compound 3, such as the anhydrous form.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S.M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Although pharmaceutically acceptable counter ions will be preferred for preparing pharmaceutical formulations, other anions are quite acceptable as synthetic intermediates. All salts described herein are preferably pharmaceutically acceptable.

In any of the embodiments herein, when Compound 2, Compound 3, Compound 4, Compound 5, or Compound 6 can be present as a salt, it will also be understood that Compound 2, Compound 3, Compound 4, Compound 5, or Compound 6 can be present as the HCl salt.

As used herein, the term “pharmaceutically acceptable excipient” includes, without limitation, any binder, filler, adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, anti-caking agent, flavor, desiccants, plasticizers, disintegrants, lubricant, polymer matrix system, and polishing agents, that have been approved by the United States Food and Drug Administration as being acceptable for use in humans or domestic animals. When excipients such as disintegrants, diluents, binders, and lubricants are recited in a formulation, it will be understood that a single compound can function as more than one kind of excipient or, alternatively, that at least one distinct compound can be included to perform each recited function, as long as each of the functions is performed in a satisfactory manner. All excipients described herein are preferably pharmaceutically acceptable.

As used herein, the phrase “in combination with one or more intra-granular excipients selected from a disintegrant, a diluent, a lubricant, or a combination thereof” means that there may only be intra-granular excipient selected from a disintegrant, a diluent or a lubricant, or there may be more than one intra-granular excipient selected from one or more disintegrants, diluents, or lubricants. As non-limiting, illustrative examples, the combination may be of one disintegrant and one lubricant; or of two different disintegrants; of two different lubricants and a diluent; or of a disintegrant, a diluent, and a lubricant.

As used herein, a series of recited values such as “Compound Z in an amount of ≤a %, b%, or c % w/w, preferably ≥d %” means that Compound Z may be in an amount of ≤a %, in an amount of ≤b %, or in an amount of ≤c %. In addition, in some embodiments, for any of these options, Compound Z is also preferably present in an amount ≥d %. As an illustrative, non-limiting example, “Compound 2 in an amount of ≤0.15%, 0.08%, or 0.05% w/w, preferably ≥0.001%” means that Compound 2 may be in an amount of ≤0.15%, in an amount of ≤0.08%, or in an amount of ≤0.05%. In addition, in some embodiments, for any of these options,

Compound 2 is also preferably present in an amount ≥0.001%. Similarly, the recitation “having an acceptance threshold ≤0.15%, 0.08%, or 0.05% w/w” indicates that the acceptance threshold is ≤0.15% w/w, ≤0.08% w/w, or ≤0.05% w/w.

### Discussion

In one embodiment, the disclosure provides a solid drug formulation comprising granulates comprising 10 to 150 mg or 15 to 150 mg or 30 to 120 mg of ulotaront HCl particles, in combination with one or more intra-granular excipients selected from a disintegrant, a diluent, a lubricant, or a combination thereof; wherein the ulotaront HCl particles have a D50 of from 37 to 98 μm.

In another embodiment, the disclosure provides ulotaront HCl particles having a D50 of from 37 to 98 μm.

In another embodiment, the disclosure provides a composition comprising ulotaront HCl and a compound selected from: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥0.001%; (c) Compound 4 in an amount of ≤0.15%, 0.08%, or 0.05% w/w, preferably ≥0.001%; or (d) Compound 5 in an amount of ≤0.60%, 0.30%, 0.10%, or 0.05% w/w, preferably ≥0.001%; wherein the percentages are based on the weight of the free base of ulotaront and the free form of the compound.

In another embodiment, the disclosure provides a solid drug formulation comprising granulates comprising 15 to 150 mg or 30 to 120 mg of ulotaront HCl particles in combination with one or more intra-granular excipients selected from a disintegrant, a diluent, a lubricant, or a combination thereof, further comprising a compound selected from: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥0.001%; (c) Compound 4 in an amount of ≤0.15%, 0.08%, or 0.05% w/w, preferably ≥0.001%; or (d) Compound 5 in an amount of ≤0.60%, 0.30%, 0.10%, or 0.05% w/w, preferably ≥0.001%; wherein the percentages are based on the weight of the free base of ulotaront and the free form of the compound.

In another embodiment, the disclosure provides ulotaront HCl comprising from 0.01% to 0.5% w/w water.

In another embodiment, the disclosure provides a solid drug formulation comprising 15 to 150 mg or 30 to 120 mg of ulotaront HCl comprising from 0.01% to 0.5% w/w water in combination with one or more excipients selected from a disintegrant, a diluent, a lubricant, or a combination thereof.

In another embodiment, the disclosure provides a method of making a pharmaceutical dosage form comprising: (a) providing a lot of ulotaront HCl comprising an amount of impurity, wherein the impurity is selected from Compound 2, Compound 3, Compound 4, Compound 5, or a salt thereof; (b) chromatographically separating the impurity from the ulotaront HCl in a sample from the lot and determining the amount of impurity in the sample; and (c) if the amount of the impurity in the sample is less than an acceptance threshold, processing the lot into one or more finished dosage forms.

In another embodiment, the disclosure provides a hemihydrate R-mandelate salt of Compound 3.

In another embodiment, the disclosure provides a method of making a hemihydrate R-mandelate salt of Compound 3 comprising crystallizing a R-mandelate salt of Compound 3 from a solution comprising acetone or acetonitrile and ≥0.7% water.

In another embodiment, the disclosure provides a method of making ulotaront HCl or a finished dosage form comprising ulotaront HCl comprising: (a) contacting a composition comprising a R-mandelate salt of ulotaront and a hemihydrate R-mandelate salt of Compound 3 with a base to form the free base of ulotaront, and (b) contacting the free base with anhydrous HCl to form ulotaront HCl.

In another embodiment, the disclosure provides a mixture comprising ulotaront HCl and Compound 2 or a salt thereof.

In another embodiment, the disclosure provides a mixture comprising ulotaront HCl and Compound 4 or a salt thereof.

In another embodiment, the disclosure provides a mixture comprising ulotaront HCl and Compound 5 or a salt thereof.

In another embodiment, the disclosure provides a hemihydrate R-mandelate salt of Compound 3 characterized by a powder x-ray diffraction pattern comprising three or more peaks, in terms of 2-theta, at 14.02±0.2°, 17.16±0.2°, 22.96±0.2°, 27.68±0.2°, 34.54±0.2°, 35.28±0.2°, and 35.7±0.2°.

In another embodiment, the disclosure provides an anhydrous R-mandelate salt of Compound 3 characterized by a powder x-ray diffraction pattern comprising three or more peaks, in terms of 2-theta, at 22.24±0.2°, 23.4±0.2°, 23.9±0.2°, and 25.86±0.2°.

In further embodiments, the formulations of the current disclosure are tablets or capsules comprising 15 to 150 or 30 to 120 mg of ulotaront HCl particles.

In further embodiments, the formulations of the current disclosure comprise granulates comprising 15 to 150 or 30 to 120 mg of ulotaront HCl particles.

In further embodiments, the formulations, particles, mixtures, or compositions of the current disclosure comprise ulotaront HCl particles having a D50 of from 37 to 98 μm.

In further embodiments, formulations, particles, mixtures, or compositions of the current disclosure comprise ulotaront HCl particles having a D10≥8 μm and a D90≤252 μm.

In further embodiments, the formulations of the current disclosure comprise granulates comprising one or more intra-granular excipients selected from a disintegrant, a diluent, a binder, a lubricant, or a combination thereof.

In further embodiments, any intra-granular excipients in the formulations of the current disclosure pass through a 30 mesh sieve.

In further embodiments, any intra-granular lubricants in the formulations of the current disclosure pass through a 60 mesh sieve.

In further embodiments, the formulations of the current disclosure comprise from 20 to 50% w/w or from 25 to 45% w/w ulotaront HCl, based on the weight of the formulation.

In further embodiments, the formulations of the current disclosure are tablets compressed from a mixture having a critical orifice diameter (“COD”) of from 9-26 mm, 16-26 mm, 9-24 mm, 16-24 mm, 9-20 mm, 20-26 mm, or 20-24 mm.

In further embodiments, the formulations of the current disclosure are tablets compressed from a mixture having a bulk density of from 0.42 to 0.48 g/cc.

In further embodiments, the formulations of the current disclosure are tablets compressed from a mixture having a tap density (250 taps) of from 0.51-0.60 g/cc.

In further embodiments, the formulations of the current disclosure are tablets compressed from a mixture having a tap density (500 taps) of from 0.52-0.61 g/cc.

In further embodiments, the formulations of the current disclosure are tablets compressed from a mixture having a Carr's Index (“CI”) of from 19.0 to 27.1.

In further embodiments, the formulations of the current disclosure are tablets compressed from a mixture having a Hausner Ratio (“HR”) of from 1.23 to 1.37.

In further embodiments, the formulations of the current disclosure are tablets compressed at a compression pressure of from 140 to 400 MPa, 160-360 MPa, 150 to 300 MPa, from 170 to 250 MPa, or from 180 to 220 MPa.

In further embodiments, the formulations of the current disclosure are tablets having a tensile strength of from 0.8 to 4.5 MPa, 1.5 to 4.0 MPa, or 1.9 to 3.3 MPa.

In further embodiments, the formulations of the current disclosure are tablets having a tensile strength based on the size of the tablet as follows:
30 mg ulotaront HCl tablet: 1.0 to 3.0 MPa, 1.8 to 2.8 MPa, or 2.1 to 2.5 MPa
60 mg ulotaront HCl tablets: 0.8 to 3.5 MPa, 1.5 to 3.0 MPa, or 1.9 to 2.3 MPa
90 mg ulotaront HCl tablets: 1.5 to 4.2 MPa, 1.7 to 3.9 MPa, or 2.9 to 3.3 MPa
120 mg ulotaront HCl tablet: 1.2 to 4.7 MPa, 2.5 to 3.5 MPa, or 2.9 to 3.2 MPa.

In further embodiments, the formulations, particles, mixtures, or compositions of the current disclosure comprise Compound 2 in an amount of ≤0.15%, 0.08%, or 0.05% w/w, preferably ≥0.001%, based on the weight of the free base of ulotaront and the free base of Compound 2.

In further embodiments, the formulations, particles, mixtures, or compositions of the current disclosure comprise Compound 3 in an amount of ≤0.30%, 0.10%, or 0.05% w/w, preferably ≥0.001%, based on the weight of the free base of ulotaront and the free base of Compound 3.

In further embodiments, the formulations, particles, mixtures, or compositions of the current disclosure comprise Compound 4 in an amount of ≤0.15%, 0.08%, or 0.05% w/w, preferably ≥0.001%, based on the weight of the free base of ulotaront and the free base of Compound 4.

In further embodiments, the formulations, particles, mixtures, or compositions of the current disclosure comprise Compound 5 in an amount of ≤0.60%, 0.30%, 0.10%, or 0.05% w/w, preferably ≥0.001%, based on the weight of the free base of ulotaront and the free base of Compound 5.

In further embodiments, the formulations, particles, mixtures, or compositions of the current disclosure comprise Compound 2 preferably in an amount of ≤0.15% w/w, based on the weight of the free base of ulotaront and the free base of Compound 2.

In further embodiments, the formulations, particles, mixtures, or compositions of the

current disclosure comprise Compound 3 preferably in an amount of ≤0.30% w/w, based on the weight of the free base of ulotaront and the free base of Compound 3.

In further embodiments, the formulations, particles, mixtures, or compositions of the current disclosure comprise Compound 4 preferably in an amount of ≤0.30% or 0.15% w/w, based on the weight of the free base of ulotaront and the free base of Compound 4.

In further embodiments, the formulations, particles, mixtures, or compositions of the current disclosure comprise Compound 5 preferably in an amount of ≤0.60% w/w, based on the weight of the free base of ulotaront and the free base of Compound 5.

In further embodiments, the formulations, particles, mixtures, or compositions of the current disclosure comprise ulotaront HCl comprising from 0.01% to 0.5% w/w water.

In further embodiments, the formulations, particles, mixtures, or compositions of the current disclosure comprise ulotaront HCl comprising from 0.015% to 0.2% w/w water.

In further embodiments, the formulations, particles, mixtures, or compositions of the current disclosure comprise ulotaront HCl comprising from 0.02% to 0.09% w/w water.

In further embodiments, the ulotaront HCl particles of the current disclosure have a D50 of from 37 to 98 μm.

In further embodiments, the ulotaront HCl particles of the current disclosure have a D10≥8 μm and a D90≤252 μm.

In further embodiments, the ulotaront HCl particles of the current disclosure are present in an isolated state.

In further embodiments, the ulotaront HCl particles of the current disclosure are present in one of the formulations of the current disclosure.

In further embodiments, the compositions of the current disclosure are provided in an isolated state.

In further embodiments, the compositions of the current disclosure are provided as a formulation of the current disclosure.

In further embodiments, the ulotaront HCl of the current disclosure is provided in an isolated state.

In further embodiments, the ulotaront HCl of the current disclosure is provided as a formulation of the current disclosure.

In further embodiments, the methods of making a pharmaceutical dosage form of the current disclosure comprise (a) providing a lot of ulotaront HCl comprising an amount of impurity, wherein the impurity is selected from Compound 2, Compound 3, Compound 4, Compound 5, or a salt thereof; (b) chromatographically separating the impurity from the ulotaront in a sample from the lot and determining the amount of impurity in the sample; and (c) if the amount of the impurity in the sample is less than an acceptance threshold, processing the lot into one or more finished dosage forms.

In further embodiments, in the methods of making a pharmaceutical dosage form of the current disclosure, the amount of the impurity is less than or equal to the acceptance threshold and the lot is processed into one or more finished dosage forms.

In further embodiments, in the methods of making a pharmaceutical dosage form of the current disclosure, the impurity is Compound 2 or a salt thereof having an acceptance threshold ≤0.15%, 0.08%, or 0.05% w/w.

In further embodiments, in the methods of making a pharmaceutical dosage form of the current disclosure, the impurity is Compound 3 or a salt thereof having an acceptance threshold ≤0.3%, 0.10%, or 0.05% w/w.

In further embodiments, in the methods of making a pharmaceutical dosage form of the current disclosure, the impurity is Compound 4 or a salt thereof having an acceptance threshold ≤0.15%, 0.08%, or 0.05% w/w.

In further embodiments, in the methods of making a pharmaceutical dosage form of the current disclosure, the impurity is Compound 5 or a salt thereof having an acceptance threshold ≤0.60%, 0.30%, 0.10%, or 0.05% w/w.

In further embodiments, in the methods of making a pharmaceutical dosage form of the current disclosure, the amount of impurity in the sample is determined by comparing an area generated by the impurity on a chromatogram of the separation to an area generated by a reference standard solution on a chromatogram obtained under like conditions.

In further embodiments, the methods of making a pharmaceutical dosage form of the current disclosure further comprise: (a) dissolving an impurity standard to form a reference standard solution; and (b) determining the amount of impurity in the sample by comparing an area generated by the impurity on a chromatogram of the separation to an area generated by a reference standard solution on a chromatogram obtained under like conditions.

In further embodiments, in the methods of making a pharmaceutical dosage form of the current disclosure that employ an impurity standard, the impurity standard is Compound 2 or a salt thereof, preferably Compound 2 free base.

In further embodiments, in the methods of making a pharmaceutical dosage form of the current disclosure that employ an impurity standard, the impurity standard is Compound 3 or a salt thereof.

In further embodiments, in the methods of making a pharmaceutical dosage form of the current disclosure that employ an impurity standard, the impurity standard is Compound 4 or a salt thereof, preferably Compound 4 HCl.

In further embodiments, in the methods of making a pharmaceutical dosage form of the current disclosure that employ an impurity standard, the impurity standard is Compound 5 or a salt thereof, preferably Compound 5 HCl.

In further embodiments, in the methods of making a pharmaceutical dosage form of the current disclosure, the impurity is selected from Compound 2, Compound 4, Compound 5, or a salt thereof, and the amount of impurity in the sample is determined by an HPLC method comprising: (a) contacting a stationary phase of a reverse phase chromatography column with the sample; (b) eluting the sample with a mobile phase such that the impurity traverses the column with a retention time that is different than the remainder of the sample; and (c) measuring the percentage composition of the sample using an ultraviolet absorptiometer at a wavelength of about 234 nm for Compound 4 or Compound 5 or 265 nm for Compound 2, wherein the mobile phase comprises a first solvent and a second solvent, the first solvent comprises aqueous 50 mM sodium perchlorate at pH 3, and the second solvent comprises methanol: acetonitrile 90:10.

In further embodiments, in the methods of making a pharmaceutical dosage form of the current disclosure, the impurity is Compound 3 or a salt thereof and the amount of impurity in the sample is determined by an HPLC method comprising: (a) contacting a stationary phase of a chromatography column with the sample; (b) eluting the sample with a mobile phase such that the impurity traverses the column with a retention time that is different than the remainder of the sample; and (c) measuring the percentage composition of the sample using an ultraviolet absorptiometer at a wavelength of about 235 nm; wherein the mobile phase comprises hexanes/ethanol/EtSO3 (85:15:0.05).

In further embodiments, the methods of the current disclosure are practiced using ulotaront HCl comprising from 0.01% to 0.5% w/w water or from 0.02% to 0.09% w/w water.

In further embodiments, the methods of the current disclosure are practiced using ulotaront HCl having a D50 from 37 to 98 μm.

In further embodiments, the methods of the current disclosure are practiced using ulotaront HCl having a D10≥8 μm and a D90≤252 μm.

In further embodiments, the methods of the current disclosure are practiced using a lot comprising Compound 2 in an amount of ≤0.05% w/w.

In further embodiments, the methods of the current disclosure are practiced using a lot comprising Compound 3 in an amount of ≤0.05% w/w.

In further embodiments, the methods of the current disclosure are practiced using a lot comprising Compound 4 in an amount of ≤0.05% w/w.

In further embodiments, the methods of the current disclosure are practiced using a lot comprising Compound 5 in an amount of ≤0.60% w/w.

In further embodiments, the methods of the current disclosure further comprise mixing all or a portion of the lot with one or more pharmaceutically acceptable excipients selected from disintegrants, diluents, binders, and lubricants, to form a mixture and processing the mixture into one or more finished dosage forms, preferably one or more tablets or capsules.

In further embodiments, the disclosure provides a finished dosage form made by any of the methods of the current disclosure.

Further embodiments relate to the hemihydrate R-mandelate salt of Compound 3, and the use of the hemihydrate R-mandelate salt of Compound 3 in the manufacture of ulotaront HCl and finished dosage forms comprising ulotaront HCl.

In some embodiments the hemihydrate R-mandelate salt of Compound 3 is characterized by a powder x-ray diffraction pattern comprising three or more peaks, in terms of 2-theta, at 14.02±0.2°, 17.16±0.2°, 22.96±0.2°, 27.68±0.2°, 34.54±0.2°, 35.28±0.2°, and 35.7±0.2°.

In some embodiments the hemihydrate R-mandelate salt of Compound 3 is characterized by its polymorphic purity. Thus, in some embodiments the hemihydrate R-mandelate salt of Compound 3 is present as a composition such as a solution or solid mixture or isolated substance, and the composition comprises no more than 50%, 25%, 10%, or 5% of the anhydrous R-mandelate salt of Compound 3.

In some embodiments, the anhydrous R-mandelate salt of Compound 3 is characterized by a powder x-ray diffraction pattern comprising three or more peaks, in terms of 2-theta, at 22.24±0.2°, 23.4±0.2°, 23.9±0.2°, and 25.86±0.2°.

In other embodiments, the disclosure provides a method of making the hemihydrate R-mandelate salt of Compound 3 comprising crystallizing a R-mandelate of Compound 3 from a solution comprising acetone or acetonitrile and ≥0.7% water.

In further embodiments, the disclosure provides a method of making ulotaront HCl or a finished dosage form comprising ulotaront HCl comprising: (a) contacting a composition comprising a R-mandelate salt of ulotaront and a hemihydrate R-mandelate salt of Compound 3 with a base to form the free base of ulotaront, and (b) contacting the free base with anhydrous HCl to form ulotaront HCl. The hemihydrate R-mandelate salt of Compound 3 preferably has one of the polymorphic purities described herein.

In further embodiments, the disclosure provides a method of making a finished dosage form, further comprising admixing the ulotaront HCl derived from the hemihydrate R-mandelate salt of Compound 3 with one or more pharmaceutically acceptable carriers selected from disintegrants, diluents, and lubricants to form a mixture and processing the mixture into a finished dosage form, preferably a tablet or capsule.

In further embodiments, the disclosure provides a mixture comprising ulotaront and Compound 2.

In further embodiments, the disclosure provides a mixture comprising ulotaront and Compound 4.

In further embodiments, the disclosure provides a mixture comprising ulotaront and

Compound 5.

In further embodiments, the disclosure provides a finished dosage form made by any of the methods or processes of the current disclosure.

### Formulations

Compositions of the present disclosure are preferably administered orally, and preferably in tablets or capsules. However, they may also be administered orally, parenterally, by inhalation, topically, rectally, nasally, buccally, sublingually, vaginally or via an implanted reservoir.

It is to be understood that in various embodiments, the pharmaceutical compositions of the present inventions comprise one or more pharmaceutically acceptable excipients, including, but not limited to, one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opacifying agents, glidants, processing aids, colorants, sweeteners, taste-masking agents, perfuming agents, flavoring agents, polishing agents, polymer matrix systems, plasticizers and other known additives to provide an elegant presentation of the drug or aid in the manufacturing of a medicament or pharmaceutical product comprising a composition of the present inventions. Examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

In various embodiments, non-limiting examples of excipients include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), hydroxypropyl cellulose, titanium dioxide, talc, calcium carbonate (e.g., granules or powder), microcrystalline cellulose, powdered cellulose, dextrates, kaolin, silicic acid, sorbitol, starch, pre-gelatinized starch, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, a syloid silica gel (AEROSIL200 (fumed silica, manufactured by Evonik), a coagulated aerosol of synthetic silica (marketed by Evonik Degussa), CAB-O-SIL (a pyrogenic silicon dioxide product sold by Cabot Co. of Boston, Mass.), colorants and mixtures thereof.

In various embodiments, the compositions are formulated with one or more pharmaceutically acceptable excipients in accordance with known and established practice. In various embodiments, the compositions described herein are referred to as formulation or medicament. Thus, in various embodiments the composition are formulated as, for example, a liquid, powder, elixir, injectable solution, or suspension. Formulations for oral use are preferred and may be provided, for instance, as tablets, caplets, or capsules, wherein the pharmacologically active ingredients are mixed with an inert solid diluent. In various embodiments, the compositions described herein are formulated as a tablet. In various embodiments, the oral dosage form is a solid oral dosage form. In various embodiments, the solid oral dosage form comprises a tablet, and in various embodiments the solid oral dosage form comprises a capsule. Tablets may also include granulating and disintegrating agents, and may be coated or uncoated. Formulations for topical use may be provided, for example as topical solutions, lotions, creams, ointments, gels, foams, patches, powders, solids, sponges, tapes, vapors, pastes or tinctures.

### ADDITIONAL EMBODIMENTS

Embodiment 1) A solid drug formulation comprising granulates comprising 30 to 120 mg of ulotaront HCl particles, in combination with one or more intra-granular excipients selected from a disintegrant, a diluent, a lubricant, or a combination thereof; wherein the ulotaront HCl particles have a D50 of from 37 to 98 μm.

Embodiment 2) The formulation of Embodiment 1, wherein the ulotaront HCl particles have a D10≥8 μm and a D90≤252 μm.

Embodiment 3) The formulation of any of Embodiments 1-2, wherein the intra-granular excipients pass through a 30 mesh sieve.

Embodiment 4) The formulation of any of Embodiments 1-3, wherein the lubricant passes through a 60 mesh sieve.

Embodiment 5) The formulation of any of Embodiments 1-4, comprising from 20 to 50% w/w or from 25 to 45% w/w ulotaront HCl, wherein the % w/w is based on the weight of the formulation.

Embodiment 6) The formulation of any of Embodiments 1-5, in the form of a tablet compressed from a mixture having one or more characteristics selected from: (a) a critical orifice diameter (“COD”) of from 9 to 26 mm, 16-26 mm, 9 to 24 mm, or 16-24 mm, (b) a bulk density of from 0.42 to 0.48 g/cc, (c) a tap density (250 taps) of from 0.51-0.60 g/cc, (d) a tap density (500 taps) of from 0.52-0.61 g/cc, (e) a Carr's Index (CI) of from 19.0 to 27.1, and (f) a Hausner Ratio (“HR”) of from 1.23 to 1.37.

Embodiment 7) The formulation of any of Embodiments 1-6, in the form of a tablet compressed at a compression pressure of from 140 to 400 MPa, 160 to 360 MPa, 150 to 300 MPa, from 170 to 250 MPa, or from 180 to 220 MPa.

Embodiment 8) The formulation of any of Embodiments 1-7, in the form of a tablet having a tensile strength of from 0.8 to 4.5 MPa, 1.5 to 4.0 MPa, or 1.9 to 3.3 MPa.

Embodiment 9) The formulation of any of Embodiments 1-8, further comprising a compound selected from: (a) Compound 2 or a salt thereof in an amount of ≤0.15%, 0.08%, or 0.05% w/w, preferably ≥0.001%; (b) Compound 3 or a salt thereof in an amount of ≤0.30%, 0.10%, or 0.05% w/w, preferably ≥0.001%; (c) Compound 4 or a salt thereof in an amount of ≤0.15%, 0.08%, or 0.05% w/w, preferably ≥0.001%; or (d) Compound 5 or a salt thereof in an amount of ≤0.60%, 0.30%, 0.10%, or 0.05% w/w, preferably ≥0.001% w/w; wherein the percentages are based on the weight of the free base of ulotaront and the free base of the compound.

Embodiment 10) The formulation of any of Embodiments 1-8, further comprising a compound selected from: (a) Compound 2 or a salt thereof preferably in an amount of ≤ 0.15% w/w; (b) Compound 3 or a salt thereof preferably in an amount of ≤0.30% w/w; (c) Compound 4 or a salt thereof preferably in an amount of ≤0.15% w/w; or (d) Compound 5 or a salt thereof preferably in an amount of ≤0.60% w/w; wherein the percentages are based on the weight of the free base of ulotaront and the free base of the compound.

Embodiment 11) The formulation of any of Embodiments 1-8, further comprising Compound 2 or a salt thereof preferably in an amount of ≤0.15% w/w based on the weight of the free base of ulotaront and the free base of Compound 2.

Embodiment 12) The formulation of any of Embodiments 1-8, further comprising Compound 4 or a salt thereof preferably in an amount of ≤0.30% w/w based on the weight of the free base of ulotaront and the free base of Compound 4.

Embodiment 13) The formulation of any of Embodiments 1-8, further comprising Compound 5 or a salt thereof preferably in an amount of ≤0.60% w/w based on the weight of the free base of ulotaront and the free base of Compound 5.

Embodiment 14) The formulation of any of Embodiments 1-8, wherein the ulotaront HCl comprises from 0.01% to 0.5% w/w water.

Embodiment 15) The formulation of any of Embodiments 1-8, wherein the ulotaront HCl comprises from 0.015% to 0.2% w/w water.

Embodiment 16) The formulation of any of Embodiments 1-8, wherein the ulotaront HCl comprises from 0.02% to 0.09% w/w water.

Embodiment 17) Ulotaront HCl particles having a D50 of from 37 to 98 μm.

Embodiment 18) The particles of Embodiment 17 having a D10≥8 μm and a D90≤252 μm.

Embodiment 19) The particles of any of Embodiments 17-18 in an isolated state.

Embodiment 20) The particles of any of Embodiments 17-19, further comprising: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥0.001%; (c) Compound 4 in an amount of ≤0.15%, 0.08%, or 0.05% w/w, preferably ≥0.001%; or (d) Compound 5 in an amount of ≤0.60%, 0.30%, 0.10%, or 0.05% w/w, preferably ≥0.001%; wherein the percentages are based on the weight of the free base of ulotaront and the free base of the compound.

Embodiment 21) The particles of any of Embodiments 17-20, wherein the ulotaront HCl comprises from 0.01% to 0.5% w/w water or from 0.02% to 0.09% w/w water.

Embodiment 22) A composition comprising ulotaront HCl and a compound selected from: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥0.001%; (c) Compound 4 in an amount of ≤0.15%, 0.08%, or 0.05% w/w, preferably ≥0.001%; or (d) Compound 5 in an amount of ≤0.60%, 0.30%, 0.10%, or 0.05% w/w, preferably ≥0.001%; wherein the percentages are based on the weight of the free base of ulotaront and the free base of the compound.

Embodiment 23) The composition of Embodiment 22 in an isolated state.

Embodiment 24) The composition of any of Embodiments 22-23, wherein the ulotaront HCl comprises from 0.01% to 0.5% w/w water or from 0.02% to 0.09% w/w water.

Embodiment 25) A solid drug formulation comprising granulates comprising 30 to 120 mg of ulotaront HCl particles in combination with one or more intra-granular excipients selected from a disintegrant, a diluent, a lubricant, or a combination thereof, further comprising a compound selected from: (a) Compound 2 in an amount of ≤0.15%, 0.08%, or 0.05% w/w, preferably ≥0.001%; (b) Compound 3 in an amount of ≤0.30%, 0.10%, or 0.05% w/w, preferably ≥0.001%; (c) Compound 4 in an amount of ≤0.15%, 0.08%, or 0.05% w/w, preferably ≥0.001%; or (d) Compound 5 in an amount of ≤0.60%, 0.30%, 0.10%, or 0.05% w/w, preferably ≥0.001%; wherein the percentages are based on the weight of the free base of ulotaront and the free base of the compound.

Embodiment 26) The formulation of Embodiment 25, wherein the ulotaront HCl comprises from 0.01% to 0.5% w/w water.

Embodiment 27) The formulation of Embodiment 25, wherein the ulotaront HCl comprises from 0.02% to 0.09% w/w water.

Embodiment 28) Ulotaront HCl comprising from 0.01% to 0.5% w/w water.

Embodiment 29) The ulotaront HCl of Embodiment 28 in an isolated state.

Embodiment 30) The ulotaront HCl of any of Embodiments 28-29, wherein the ulotaront HCl comprises from 0.02% to 0.09% w/w water.

Embodiment 31) A solid drug formulation comprising 30 to 120 mg of ulotaront HCl comprising from 0.01% to 0.5% w/w water in combination with one or more excipients selected from a disintegrant, a diluent, a lubricant, or a combination thereof.

Embodiment 32) The formulation of Embodiment 31 comprising the ulotaront HCl as granulates wherein the ulotaront HCl comprises from 0.01% to 0.5% w/w water in combination with one or more intra-granular excipients selected from a disintegrant, a diluent, a lubricant, or a combination thereof.

Embodiment 33) The formulation of any of Embodiments 31-32, wherein the ulotaront HCl comprises from 0.02% to 0.09% w/w water.

Embodiment 34) A method of making a pharmaceutical dosage form comprising: (a) providing a lot of ulotaront HCl comprising an amount of impurity, wherein the impurity is selected from Compound 2, Compound 3, Compound 4, and Compound 5, or a salt thereof; (b) chromatographically separating the impurity from the ulotaront in a sample from the lot and determining the amount of impurity in the sample; and (c) if the amount of the impurity in the sample is less than an acceptance threshold, processing the lot into one or more finished dosage forms.

Embodiment 35) The method of Embodiment 34, wherein the amount of the impurity is less than or equal to the acceptance threshold.

Embodiment 36) The method of any of Embodiments 34-35, wherein: (a) the impurity is Compound 2 or a salt thereof having an acceptance threshold ≤0.15%, 0.08%, or 0.05% w/w; (b) the impurity is Compound 3 or a salt thereof having an acceptance threshold ≤0.3%, 0.10%, or 0.05% w/w; (c) the impurity is Compound 4 or a salt thereof having an acceptance threshold ≤0.15%, 0.08%, or 0.05% w/w; or (d) the impurity is Compound 5 or a salt thereof having an acceptance threshold ≤0.60%, 0.30%, 0.10%, or 0.05% w/w; wherein the percentages are based on the weight of the free base of ulotaront and the free base of the compound.

Embodiment 37) The method of any of Embodiments 34-36, wherein the amount of impurity in the sample is determined by comparing an area generated by the impurity on a chromatogram of the separation to an area generated by a reference standard solution on a chromatogram obtained under like conditions.

Embodiment 38) The method of any of Embodiments 34-37, further comprising: (a) dissolving an impurity standard to form a reference standard solution; and (b) determining the amount of impurity in the sample by comparing an area generated by the impurity on a chromatogram of the separation to an area generated by a reference standard solution on a chromatogram obtained under like conditions; wherein the impurity standard is selected from: Compound 2 or a salt thereof, preferably Compound 2 free base; Compound 3 or a salt thereof; Compound 4 or a salt thereof, preferably Compound 4 HCl; Compound 5 or a salt thereof, preferably Compound 5 HCl; or Compound 6 or a salt thereof, preferably Compound 6 HCl.

Embodiment 39) The method of any of Embodiments 34-38, wherein the impurity is selected from Compound 2, Compound 4, Compound 5, or a salt thereof, and the amount of impurity in the sample is determined by an HPLC method comprising: (a) contacting a stationary phase of a reverse phase chromatography column with the sample; (b) eluting the sample with a mobile phase such that the impurity traverses the column with a retention time that is different than the remainder of the sample; and (c) measuring the percentage composition of the sample using an ultraviolet absorptiometer at a wavelength of about 234 nm for Compound 4 or Compound 5 or 265 nm for Compound 2; wherein the mobile phase comprises a first solvent and a second solvent, the first solvent comprises aqueous 50 mM sodium perchlorate at pH 3, and the second solvent comprises methanol: acetonitrile 90:10.

Embodiment 40) The method of any of Embodiments 34-38, wherein the impurity is Compound 3 or a salt thereof and the amount of impurity in the sample is determined by an HPLC method comprising: (a) contacting a stationary phase of a chromatography column with the sample; and (b) eluting the sample with a mobile phase such that the impurity traverses the column with a retention time that is different than the remainder of the sample; and (c) measuring the percentage composition of the sample using an ultraviolet absorptiometer at a wavelength of about 235 nm; wherein the mobile phase comprises hexanes/ethanol/EtSO3 (85:15:0.05).

Embodiment 41) The method of any of Embodiments 34-40, wherein the ulotaront HCl comprises from 0.01% to 0.5% w/w water or from 0.02% to 0.09% w/w water.

Embodiment 42) The method of any of Embodiments 34-41, wherein the ulotaront HCl has a D50 from 37 to 98 μm.

Embodiment 43) The method of any of Embodiments 34-42, wherein the ulotaront HCl has a D10≥8 μm and a D90≤252 μm.

Embodiment 44) The method of any of Embodiments 34-43, wherein the lot comprises Compound 2 or a salt thereof in an amount of ≤0.05% w/w wherein the percentages are based on the weight of the free base of ulotaront and the free base of Compound 2.

Embodiment 45) The method of any of Embodiments 34-44, wherein the lot comprises Compound 3 or a salt thereof in an amount of ≤0.05% w/w wherein the percentages are based on the weight of the free base of ulotaront and the free base of Compound 3.

Embodiment 46) The method of any of Embodiments 34-45, wherein the lot comprises Compound 4 or a salt thereof in an amount of ≤0.05% w/w wherein the percentages are based on the weight of the free base of ulotaront and the free base of Compound 4.

Embodiment 47) The method of any of Embodiments 34-46, wherein the lot comprises Compound 5 or a salt thereof in an amount of ≤0.60% w/w wherein the percentages are based on the weight of the free base of ulotaront and the free base of Compound 5.

Embodiment 48) The method of any of Embodiments 34-47, further comprising mixing all or a portion of the lot with one or more pharmaceutically acceptable excipients selected from disintegrants, diluents, and lubricants, to form a mixture and processing the mixture into one or more finished dosage forms, preferably one or more tablets or capsules.

Embodiment 49) A finished dosage form made by the method of any of Embodiments 34-48.

Embodiment 50) A hemihydrate R-mandelate salt of Compound 3.

Embodiment 51) The hemihydrate of Embodiment 50 having a polymorphic purity relative to the anhydrous R-mandelate salt of Compound 3 of greater than 50%, greater than 75%, greater than 90%, or greater than 95%.

Embodiment 52) A method of making a hemihydrate R-mandelate salt of Compound 3 comprising crystallizing a R-mandelate salt of Compound 3 from a solution comprising acetone or acetonitrile and ≥0.7% water.

Embodiment 53) A method of making ulotaront HCl or a finished dosage form comprising ulotaront HCl comprising: (a) contacting a composition comprising a R-mandelate salt of ulotaront and a hemihydrate R-mandelate salt of Compound 3 with a base to form the free base of ulotaront, and (b) contacting the free base of ulotaront with anhydrous HCl to form ulotaront HCl.

Embodiment 54) The method of Embodiment 53, further comprising admixing the ulotaront HCl with one or more pharmaceutically acceptable carriers selected from disintegrants, diluents, and lubricants to form a mixture and processing the mixture into a finished dosage form, preferably a tablet or capsule.

Embodiment 55) A finished dosage form made by the process of any of Embodiments 52-54.

Embodiment 56) A mixture comprising ulotaront HCl and Compound 2 or a salt thereof.

Embodiment 57) A mixture comprising ulotaront HCl and Compound 4 or a salt thereof.

Embodiment 58) A mixture comprising ulotaront HCl and Compound 5 or a salt thereof.

Embodiment 59) A hemihydrate R-mandelate salt of Compound 3 characterized by a powder x-ray diffraction pattern comprising three or more peaks, in terms of 2-theta, at 14.02±0.2°, 17.16±0.2°, 22.96±0.2°, 27.68±0.2°, 34.54±0.2°, 35.28±0.2°, and 35.7±0.2°.

Embodiment 60) An anhydrous R-mandelate salt of Compound 3 characterized by a powder x-ray diffraction pattern comprising three or more peaks, in terms of 2-theta, at 22.24±0.2°, 23.4±0.2°, 23.9±0.2°, and 25.86±0.2°.

### EXAMPLES

In the following examples, efforts have been made to ensure accuracy with respect to numbers (e.g., amounts, temperature, etc.) but some errors and deviations should be accounted for. The following examples are put forth so as to provide those of ordinary skill in the art with a complete disclosure and description of how the methods claimed herein are made and evaluated and are intended to be purely exemplary of the disclosure and are not intended to limit the scope of what the inventors regard as their invention.

### Example 1: Evaluation of the Impact of API Particle Size on Flowability and Tabletability of Ulotaront HCl Blends With a Compaction Simulator

API particle size distribution can be a critical factor for tablet stability, and a critical process parameter for the compression step during tableting. Therefore, it is very important to understand the impact of API particle size on tablet properties from a drug product quality perspective and to set appropriate API size specifications.

All the prepared blends were evaluated for flowability including Carr's Index (CI), Hausner Ratio (HR) and critical orifice diameter (COD). CI and HR were calculated by the following equations:

$$CI = ( Tap density - Bulk density ) / Tap density * 100$$

$$HR = Tap density / Bulk density$$

Tensile strength (TS) of round flat tablets is calculated by the following equation:

$$TS ( MPa ) = 2 * F / π * D * H$$

and TS of round biconvex tablet is calculated by the following equation:

$$TS ( MPa ) = 10 * F / π {D}^{2} * ( 2.84 * H / D - 0.126 * H / W + 3.15 * w / d + 0.01 ) )$$

where F is breaking force (N), D is tablet diameter (mm), H is tablet thickness (mm), and W is the tablet central cylinder thickness (tablet wall height) (mm) (USP <1217>).

A compaction simulator was used in this study to simulate the compression process with a limited amount of powder. Two compression simulation modes were used in this study: a two stage force control mode (FC) at target dwell time of 120 ms, and a compaction simulation mode (CS) simulating at press speed of 40 rpm. Tablets compressed at both modes were characterized to generate the tabletability profile and to predict potential scale-up risk for each formulation.

The active pharmaceutical ingredients (“APIs”) used for this study are summarized in Table 1. Compositions of tablets from Tablet Lots 1 and 2 are summarized in Table 2 and Table 3, respectively. Except for magnesium stearate, all ingredients including API were dispensed and sieved through 30 mesh before charging into a 500 mL plastic bottle. These ingredients were blended for 4 min at blender setting 46 with a Turbula® (Willy A. Bachofen A G, Mottenz, Switzerland) mixer. After mixing, magnesium stearate was sieved through 60 mesh before weighing and charging into the bottle to eliminate material loss during sieving. The blend was lubricated for 1 min at blender setting 46 with the Turbula® mixer.

For each blend from Tablet Lot 1, a portion was compacted with a concave, 3/8″ round tooling. The remaining blends and all blends from Tablet Lot 2 were compacted with a flat face, ⅜″ round tooling. The target tablet weight was 300 mg. 150.2, 200.7, 249.8 and 300.3 MPa were set as the target compression pressures, corresponding to 10.7, 14.3, 17.8 and 21.4 kN of compression force. In FC mode, 120 ms was set as the target dwell time. In CS mode, press speed of 40 rpm was simulated. The dwell time for this condition was approximately 10 ms. All blends were measured for bulk density, tap density and COD, and CI and HR were calculated. All tablets were characterized for tablet weight, thickness and hardness, and tensile strength was calculated based on these results.

TABLE 1
Characteristics of Ulotaront HCl API Used in This Study

Particle Size Distribution

| API Lot | | D10 | D50 | D90 | |
|---|---|---|---|---|---|
| No. | Milling | [μm] | [μm] | [μm] | SPAN* |
| API-1 | − | 36 | 68 | 123 | 1.28 |
| API-2 | − | 69 | 149 | 335 | 1.79 |
| API-3 | − | 69 | 157 | 376 | 1.96 |
| API-4 | − | 74 | 189 | 382 | 1.63 |
| API-5 | − | 72 | 108 | 161 | 0.82 |
| API-6 | + | 27 | 65 | 120 | 1.43 |
| API-7 | + | 14 | 65 | 138 | 1.91 |
| API-8 | + | 39 | 108 | 236 | 1.82 |
| API-9 | + | 10 | 54 | 130 | 2.22 |
| API-10 | + | 33 | 106 | 234 | 1.90 |

*SPAN is calculated as *SPAN is calculated as (D90 − D10) divided by D50.

TABLE 2
Tablet Composition (Tablet Lot 1)

| | Blend 1 | Blend 2 | Blend 3 | Blend 4 | Blend 5 | Blend 6 | Blend 7 | Blend 8 |
|---|---|---|---|---|---|---|---|---|
| | API-1 | API-1 | API-2 | API-2 | API-3 | API-3 | API-4 | API-4 |
| Ulotaront HCl, | 30.00 | 40.00 | 30.00 | 40.00 | 30.00 | 40.00 | 30.00 | 40.00 |
| API | (90.0) | (120.0) | (90.0) | (120.0) | (90.0) | (120.0) | (90.0) | (120.0) |
| API D50 [μm] | 68 | 68 | 149 | 149 | 157 | 157 | 189 | 189 |
| Microcrystalline | 48.34 | 46.16 | 48.34 | 46.16 | 48.34 | 46.16 | 48.34 | 46.16 |
| Cellulose | (145.0) | (138.5) | (145.0) | (138.5) | (145.0) | (138.5) | (145.0) | (138.5) |
| Mannitol | 18.21 | 10.09 | 18.21 | 10.09 | 18.21 | 10.09 | 18.21 | 10.09 |
| | (54.63) | (30.28) | (54.63) | (30.28) | (54.63) | (30.28) | (54.63) | (30.28) |
| Sodium Starch | 2.86 | 3.14 | 2.86 | 3.14 | 2.86 | 3.14 | 2.86 | 3.14 |
| Glycolate | (8.572) | (9.428) | (8.572) | (9.428) | (8.572) | (9.428) | (8.572) | (9.428) |
| Magnesium | 0.59 | 0.61 | 0.59 | 0.61 | 0.59 | 0.61 | 0.59 | 0.61 |
| Stearate | (1.757) | (1.843) | (1.757) | (1.843) | (1.757) | (1.843) | (1.757) | (1.843) |
| Total | 100 | 100 | 100 | 100 | 100 | 100 | 100 | 100 |
| | (300) | (300) | (300) | (300) | (300) | (300) | (300) | (300) |

TABLE 3
Tablet Composition (Tablet Lot 2)

| | Blend | Blend | Blend | Blend | Blend | Blend | Blend | Blend | Blend | Blend | Blend | Blend |
|---|---|---|---|---|---|---|---|---|---|---|---|---|
| | 1 | 2 | 3 | 4 | 5 | 6 | 7 | 8 | 9 | 10 | 11 | 12 |
| | API-6 | API-6 | API-5 | API-5 | API-7 | API-7 | API-8 | API-8 | API-9 | API-9 | API-10 | API-10 |
| Ulotaront HCl | 30.00 | 40.00 | 30.00 | 40.00 | 30.00 | 40.00 | 30.00 | 40.00 | 30.00 | 40.00 | 30.00 | 40.00 |
| API | (90.0) | (120.0) | (90.0) | (120.0) | (90.0) | (120.0) | (90.0) | (120.0) | (90.0) | (120.0) | (90.0) | (120.0) |
| API D50 [μm] | 65 | 65 | 108 | 108 | 65 | 65 | 108 | 108 | 54 | 54 | 106 | 106 |
| Microcrystalline | 48.34 | 46.16 | 48.34 | 46.16 | 48.34 | 46.16 | 48.34 | 46.16 | 48.34 | 46.16 | 48.34 | 46.16 |
| Cellulose | (145.0) | (138.5) | (145.0) | (138.5) | (145.0) | (138.5) | (145.0) | (138.5) | (145.0) | (138.5) | (145.0) | (138.5) |
| Mannitol | 18.21 | 10.09 | 18.21 | 10.09 | 18.21 | 10.09 | 18.21 | 10.09 | 18.21 | 10.09 | 18.21 | 10.09 |
| | (54.63) | (30.28) | (54.63) | (30.28) | (54.63) | (30.28) | (54.63) | (30.28) | (54.63) | (30.28) | (54.63) | (30.28) |
| Sodium Starch | 2.86 | 3.14 | 2.86 | 3.14 | 2.86 | 3.14 | 2.86 | 3.14 | 2.86 | 3.14 | 2.86 | 3.14 |
| Glycolate | (8.572) | (9.428) | (8.572) | (9.428) | (8.572) | (9.428) | (8.572) | (9.428) | (8.572) | (9.428) | (8.572) | (9.428) |
| Magnesium | 0.59 | 0.61 | 0.59 | 0.61 | 0.59 | 0.61 | 0.59 | 0.61 | 0.59 | 0.61 | 0.59 | 0.61 |
| Stearate | (1.757) | (1.843) | (1.757) | (1.843) | (1.757) | (1.843) | (1.757) | (1.843) | (1.757) | (1.843) | (1.757) | (1.843) |
| Total | 100 | 100 | 100 | 100 | 100 | 100 | 100 | 100 | 100 | 100 | 100 | 100 |
| | (300) | (300) | (300) | (300) | (300) | (300) | (300) | (300) | (300) | (300) | (300) | (300) |

COD, bulk density, tap density (tapped 250 and 500 times), Cl, and HR are summarized in Table 4 for Tablet Lot Land Table 5 for Tablet Lot 2, respectively, The powder nowability decreases when API particle size decreases, and the flowability difference was not significant between blends at 30% w/w and 40% w/w drug loading.

TABLE 4
Bulk/Tap Density and Flowability for Blends 1-8, Tablet Lot 1

| Blend No. | 1 | 2 | 3 | 4. | 5 | 6 | 7 | 8 |
|---|---|---|---|---|---|---|---|---|
| API D10 [μm] | 36 | 36 | 69 | 69 | 69 | 69 | 74 | 74 |
| API D50 [μm] | 68 | 68 | 149 | 149 | 157 | 157 | 189 | 189 |
| API D90 [μm] | 123 | 123 | 335 | 335 | 376 | 376 | 382 | 382 |
| API loading % | 30 | 40 | 30 | 40 | 30 | 40 | 30 | 40 |
| COD [mm] | 14 | 16 | 10 | 12 | 9 | 10 | 9 | 10 |
| Bulk density [g/cc] | 0.42 | 0.43 | 0.44 | 0.46 | 0.45 | 0.47 | 0.45 | 0.47 |
| Tap density - 1 [g/cc]*¹ | 0.53 | 0.57 | 0.55 | 0.57 | 0.54 | 0.57 | 0.55 | 0.58 |
| Tap density - 2 [g/cc]*² | 0.54 | 0.59 | 0.56 | 0.58 | 0.56 | 0.58 | 0.56 | 0.59 |
| ClI*³ | 22.2 | 27.1 | 21.4 | 20.7 | 19.6 | 19.0 | 19.6 | 20.3 |
| HR*³ | 1.29 | 1.37 | 1.27 | 1.26 | 1.24 | 1.23 | 1.24 | 1.26 |

*¹Tapped 250 times
*²Tapped 500 times
*³Tap density - 2 was used for the calculations.

TABLE 5
Bulk/Tap Density and Flowability for Blends 1-12, Tablet Lot 2

| Blend No. | 1 | 2 | 3 | 4 | 5 | 6 | 7 | 8 | 9 | 10 | 11 | 12 |
|---|---|---|---|---|---|---|---|---|---|---|---|---|
| API D10 [μm] | 27 | 27 | 39 | 39 | 14 | 14 | 39 | 39 | 10 | 10 | 33 | 33 |
| API D50 [μm] | 65 | 65 | 108 | 108 | 65 | 65 | 108 | 108 | 54 | 54 | 106 | 106 |
| API D90 [μm] | 120 | 120 | 236 | 236 | 138 | 138 | 236 | 236 | 130 | 130 | 234 | 234 |
| APIloading % | 30 | 40 | 30 | 40 | 30 | 40 | 30 | 40 | 30 | 40 | 30 | 40 |
| COD [mm] | 16 | 16 | 10 | 10 | 18 | 18 | 12 | 10 | 18 | 20 | 12 | 14 |
| Bulk density [g/cc] | 0.44 | 0.45 | 0.42 | 0.42 | 0.45 | 0.46 | 0.45 | 0.47 | 0.45 | 0.46 | 0.46 | 0.48 |
| Tap density - 1 [g/cc]*¹ | 0.56 | 0.59 | 0.51 | 0.52 | 0.57 | 0.60 | 0.57 | 0.59 | 0.57 | 0.60 | 0.57 | 0.60 |
| Tap density - 2 [g/cc]*² | 0.57 | 0.60 | 0.52 | 0.53 | 0.58 | 0.61 | 0.58 | 0.60 | 0.59 | 0.61 | 0.58 | 0.60 |
| CI*³ | 22.8 | 25.0 | 19.2 | 20.8 | 22.4 | 24.6 | 22.4 | 21.7 | 23.7 | 24.6 | 20.7 | 20.0 |
| HR*³ | 1.30 | 1.33 | 1.24 | 1.26 | 1.29 | 1.33 | 1.29 | 1.28 | 1.31 | 1.33 | 1.26 | 3.25 |

*¹Tapped 250 times
*²Tapped 500 times
*³Tap density - 2 was used for the calculations.

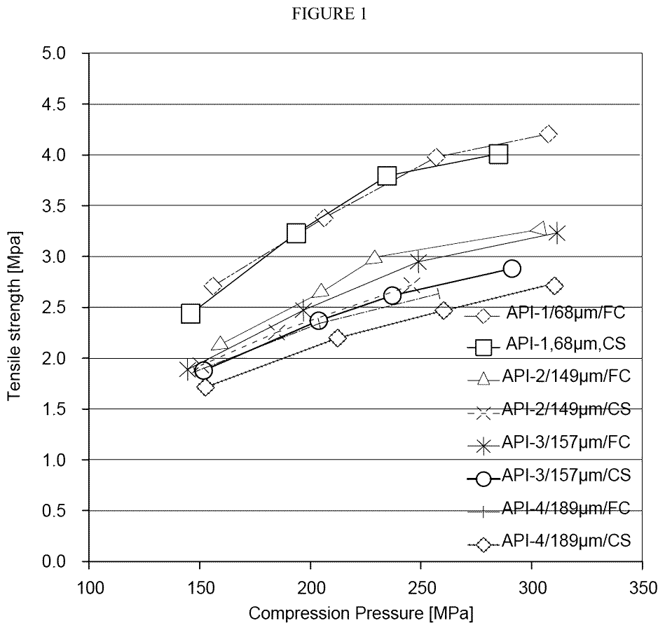

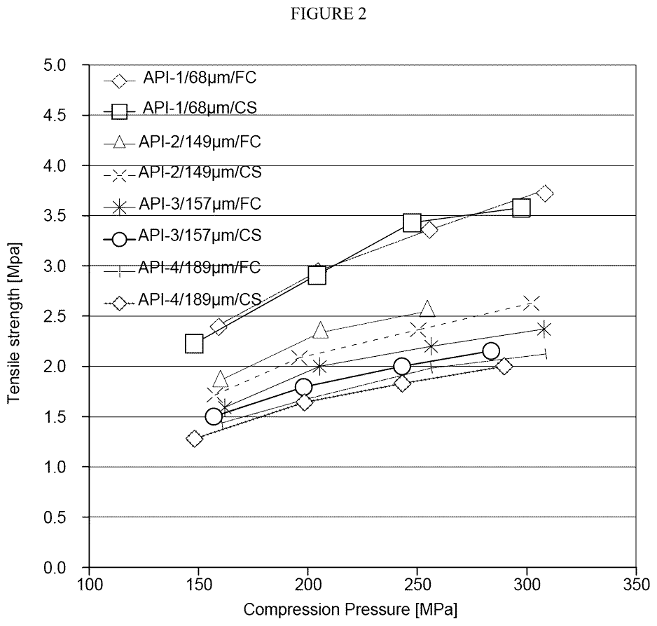

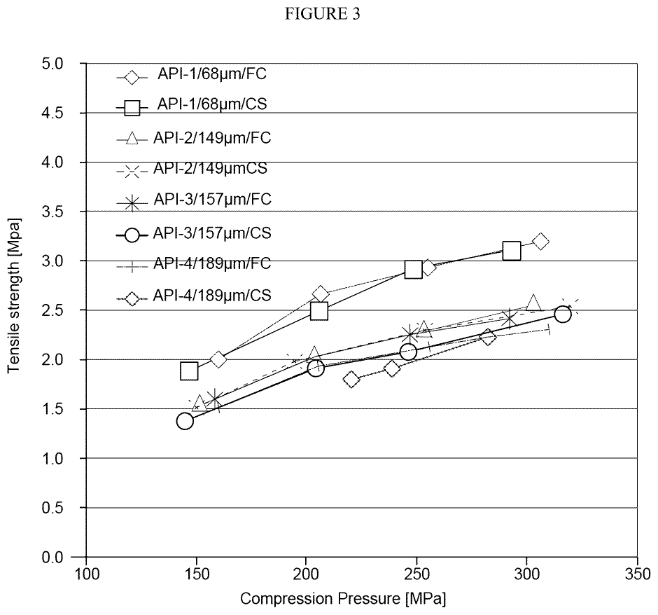

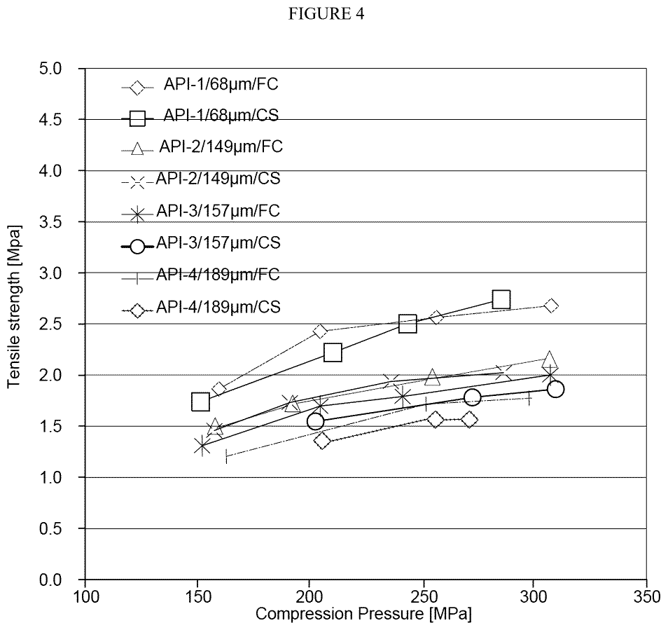

Compression profiles for the 8 blends of Tablet Lot 1 under 2 stage PC mode (Target dwell time of 120 ms) and CS mode simulating Fette 3090 at 40 rpm press speed are summarized in FIG. 1 (flat tablets at 30% w/w API loading); FIG. 2 (flat tablets at 40% w/w API loading); FIG. 3 (biconvex tablets at 30% w/w API loading); and FIG. 4 (biconvex tablets at 40% w/w API loading).

As API particle size decreased, tabletability improved for both flat and biconvex tablets. At the same drug loading, blends 1 and 2 containing API lot API-1 (D50=68 μm) had much better tabletability than the other blends. Tabletability for biconvex tablets was lower than the tabletability for flat ones. For biconvex tablets at 40% w/w API loading. only blend 2 (D50=68 μm) achieved TS of 2 MPa at 200 MPa compression pressures.

API particle size reduction by milling improved tabletability, and all blends containing milled API at 30% w/w and 40% w/w API loading achieved TS of 2 MPa at 200 MPa compression pressures. Based on preliminary statistical analysis, API particle size and compression pressure are significant factors for tablet tensile strength. Ulotaront HCl API particle size reduction through milling improved tabletability.

### Example 2: Establishment of Acceptable Ulotaront HCl Particle Size Distribution Range

In order to establish an acceptable range for ulotaront HCl particle size distribution (PSD), tablets were manufactured using drug substance lots of widely varying PSD. and processability and quality attributes of the drug product were evaluated. The edges of the size range were chosen to encompass all milled drug substance lots utilized during the clinical phase, as well as anticipated future drug substance manufactured during the commercial phase.

Milling conditions outside of the normal operating range were used to obtain lots with significantly smaller or larger particle size than that produced by milling within the normal operating range. The particle size was measured before and after milling using laser diffraction.

TABLE 6
PSD for Ulotaront Lots Used in the
Drug Substance PSD Range Study

| Tablet Batch | API | Dv(10) | Dv(50) | Dv(90) | PSD |
|---|---|---|---|---|---|
| Number | Lot Number | (μm)^{a} | (μm)^{a} | (μm)^{a} | Range |
| Tablet Batch 1 | API Lot 1 | 22 | 98 | 252 | High |
| Tablet Batch 2 | API Lot 2 | 14 | 67 | 218 | Target |
| Tablet Batch 3 | API Lot 3 | 8 | 37 | 120 | Low |

a All values shown are an average of three individual measurements.

Three batches of ulotaront 75 mg tablets using the drug substance with the PSD described in Table 6 were manufactured at a pilot scale, using essentially the same formulation described in Example 1. Blending and dry granulation were done following the established process and tablets were compressed using oval-shaped tooling used for clinical tablets, at target 300 mg unit weight proposed for the commercial 75 mg tablet strength. All three batches were successfully processed without any technical issues. The compaction profiles generated on a rotary tablet press are shown in FIG. 5 and demonstrate similar behavior for the three batches. As summarized in Table 7 the in-process data as well as blend uniformity (BU) and stratified content uniformity (CU) and dissolution properties were similar for all three batches and within defined specifications.

TABLE 7
Summary of In-Process Tests and Additional Characterization Data
for Drug Product Batches from the Drug Substance PSD Range Study

| Tablet Batch Number | Tablet Batch 1 | Tablet Batch 2 | Tablet Batch 3 |
|---|---|---|---|
| API Lot Number | API Lot 1 | API Lot 2 | API Lot 3 |
| Tablet weight (mg) | 299.1/0.5 | 301.5/0.7 | 300.0/0.7 |
| Mean/% RSD | | | |
| Tablet hardness (kP) | 16.9/4.4 | 16.9/5.2 | 17.6/5.0 |
| Mean/% RSD | | | |
| Friability (%) | 0.2 | 0.1 | 0.1 |
| BU (% LC) Mean/SD/n | 98.3/0.4/10 | 98.5/1.2/10 | 99.6/2.6/10 |
| Stratified CU (% LC) | 100.5/1.0/54 | 100.9/0.9/54 | 100.2/1.1/60 |
| Mean/SD/n | | | |
| % Dissolved in 15/30 min | | | |
| Lowest Hardness | 99.2/99.3 | 99.8/99.8 | 100.6/100.7 |
| Highest Hardness | 98.8/99.6 | 99.8/100.6 | 98.1/98.4 |

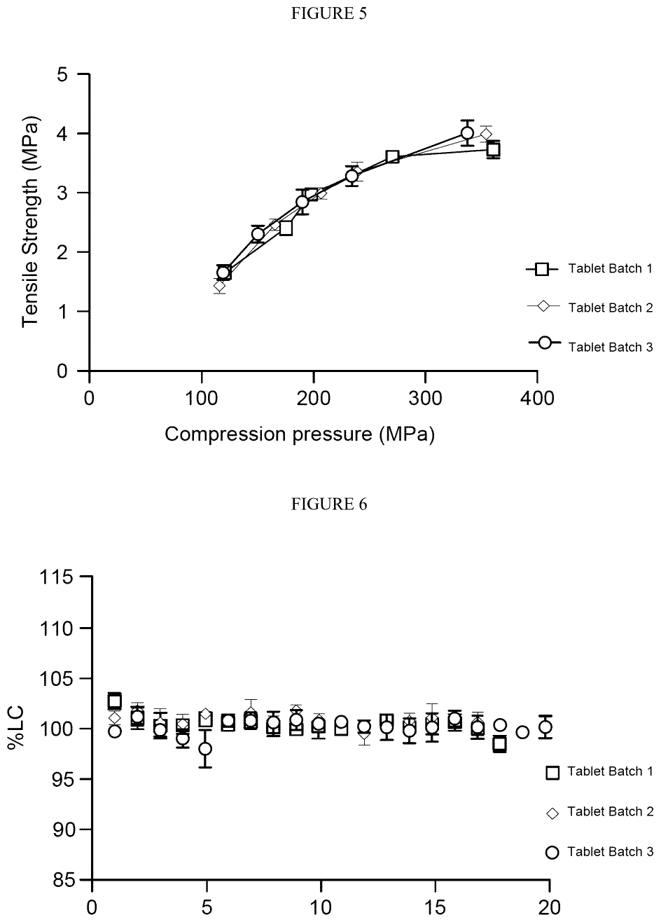

No correlation was found between tablet attributes and the PSD of the corresponding drug substance lot used. FIG. 6 presents the stratified content uniformity test results performed on three tablets at 18-20 locations throughout tablet compression for each of the three batches and demonstrates low variability and no evidence of segregation. The study demonstrated that the milled ulotaront HCl particle size within the studied range (Table 6) has no effect on tablet processability or tablet quality attributes.

### Example 3: Manufacture of ulotaront HCl Having High Chiral Purity via the Hemihydrate R-Mandelate Salt of Compound 3

As described in US 2019/0256525 A1, the manufacture of ulotaront HCl can proceed through the (R)-mandelate intermediary. R-mandelic acid mediated diastereomeric resolution of free base Compound 6 is shown in the following reaction scheme.

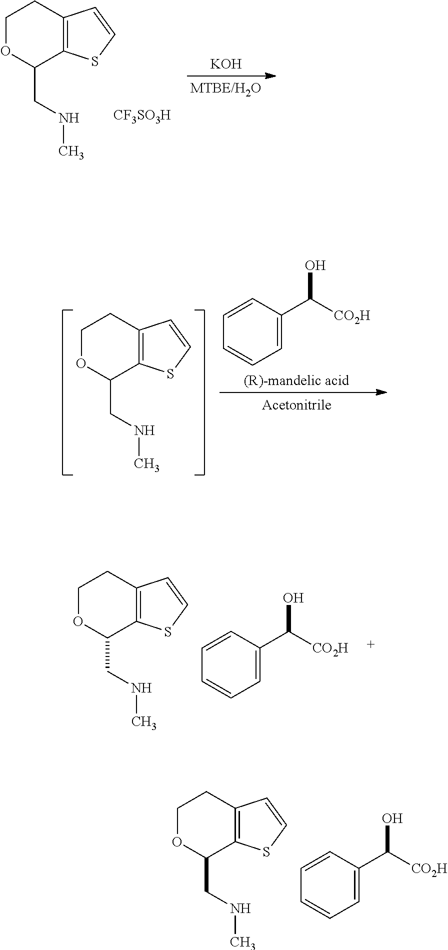

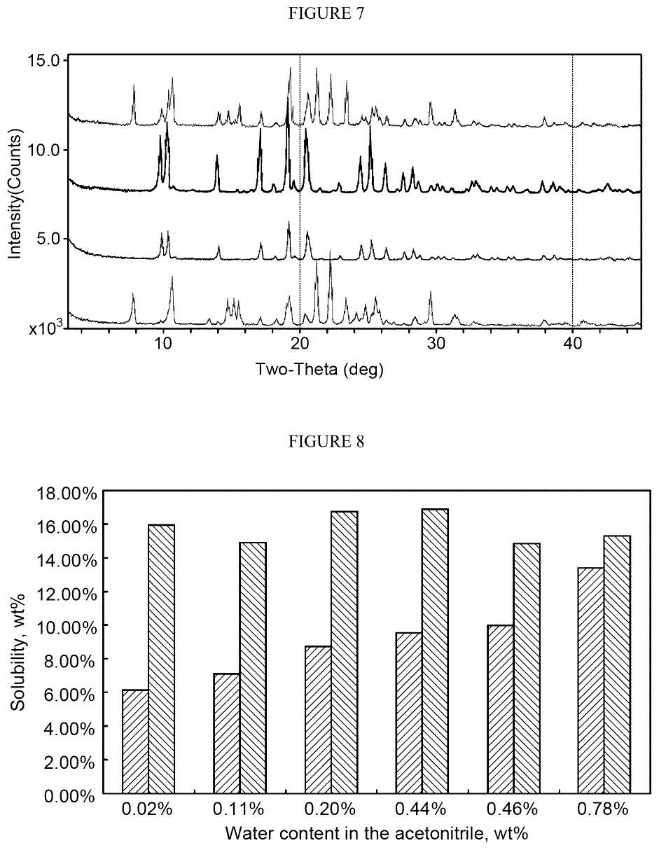

The crude R-mandelate salt of Compound 1 is produced with the R-mandelate salt of Compound 3 as a major impurity. DVS, XRD, DSC and Raman characterization of the isolated R-mandelate salts of Compound 3 and Compound 1 has demonstrated that the R-mandelate salt of Compound 3 exists in both an anhydrous form and a hemihydrate form, while the R-mandelate salt of Compound 1 does not. It has further been demonstrated that the anhydrous Compound 3 (mandelate salt) forms when the R-mandelate salt of Compound 3 is dried in the oven at 40° C. for a long period of time, i.e., a week; and that the hemihydrate Compound 3 (mandelate salt) forms when the R-mandelate salt of Compound 3 is exposed to high humidity conditions for a long period of time, i.e., a couple of days. FIG. 7 depicts XRD patterns of the anhydrous and hemihydrate R-mandelate salts of Compound 3. Table 8 is an XRD peak comparison of the anhydrous and hemihydrate forms of the R-mandelate salt of compound 3.

TABLE 8

| Anhydrous | | | Hemihydrate | | |
|---|---|---|---|---|---|
| | | Relative Height, | | | Relative |
| 2θ, ° | Intensity | % | 2θ, ° | Intensity | Height, % |
| 7.8 | 5173.333 | 73.0% | 9.86 | 2553.333 | 67.9% |
| 10.6 | 4233.333 | 59.7% | 10.34 | 2908.333 | 77.3% |
| 13.38 | 943.3333 | 13.3% | 10.88 | 506.6667 | 13.5% |
| 14.72 | 2415 | 34.1% | 14.02 | 1508.333 | 40.1% |
| 15.22 | 2548.333 | 36.0% | 15.5 | 296.6667 | 7.9% |
| 15.56 | 3030 | 42.7% | 16.56 | 306.6667 | 8.2% |
| 18.3 | 943.3333 | 13.3% | 17.16 | 1795 | 47.7% |
| 19.04 | 1240 | 17.5% | 18.22 | 530 | 14.1% |
| 19.3 | 2803.333 | 39.5% | 19.22 | 3760 | 100.0% |
| 20.4 | 1515 | 21.4% | 19.62 | 551.6667 | 14.7% |
| 21.22 | 5655 | 79.8% | 20.56 | 2785 | 74.1% |
| 22.24 | 7088.333 | 100.0% | 21.6 | 330 | 8.8% |
| 23.4 | 4941.667 | 69.7% | 22.96 | 615 | 16.4% |
| 23.9 | 793.3333 | 11.2% | 24.5 | 1606.667 | 42.7% |
| 24.16 | 1411.667 | 19.9% | 25.28 | 2075 | 55.2% |
| 24.78 | 1850 | 26.1% | 26.36 | 1293.333 | 34.4% |
| 25.54 | 2100 | 29.6% | 27.22 | 301.6667 | 8.0% |
| 25.86 | 1781.667 | 25.1% | 27.68 | 926.6667 | 24.6% |
| 26.44 | 483.3333 | 6.8% | 28.34 | 1180 | 31.4% |
| 26.94 | 531.6667 | 7.5% | 28.8 | 655 | 17.4% |
| 28.52 | 1186.667 | 16.7% | 29.7 | 476.6667 | 12.7% |
| 29.58 | 3573.333 | 50.4% | 30.18 | 541.6667 | 14.4% |
| 31.36 | 2380 | 33.6% | 30.56 | 523.3333 | 13.9% |
| 31.6 | 1090 | 15.4% | 31.26 | 390 | 10.4% |
| 32.78 | 488.3333 | 6.9% | 32.34 | 295 | 7.8% |
| 33.94 | 361.6667 | 5.1% | 32.72 | 596.6667 | 15.9% |
| 37.88 | 680 | 9.6% | 33.02 | 648.3333 | 17.2% |
| 38.96 | 445 | 6.3% | 34.14 | 406.6667 | 10.8% |
| 39.46 | 660 | 9.3% | 34.54 | 393.3333 | 10.5% |
| 40.7 | 668.3333 | 9.4% | 35.28 | 435 | 11.6% |
| 41.04 | 673.3333 | 9.5% | 35.7 | 493.3333 | 13.1% |
| 41.5 | 391.6667 | 5.5% | 36.74 | 323.3333 | 8.6% |
| 43 | 371.6667 | 5.2% | 37.88 | 668.3333 | 17.8% |
| 44.7 | 411.6667 | 5.8% | 38.66 | 666.6667 | 17.7% |
| | | | 39.1 | 381.6667 | 10.2% |
| | | | 39.76 | 275 | 7.3% |
| | | | 40.54 | 313.3333 | 8.3% |
| | | | 42.06 | 348.3333 | 9.3% |
| | | | 42.68 | 506.6667 | 13.5% |
| | | | 43.22 | 321.6667 | 8.6% |
| | | | 43.56 | 263.3333 | 7.0% |
| | | | 44.14 | 390 | 10.4% |

In order to understand and interpret the effects of the solid-liquid thermodynamics phase equilibria of the anhydrous and hemihydrate R-mandelate salts of Compound 3, solubilities in acetonitrile with different water content were evaluated. The solubility of the R-mandelate salt of anhydrous Compound 3, in the presence of the R-mandelate salt of Compound 1, in acetonitrile showed strong dependency on the water content. Its solubility increased from 6.1% to 13.41% as the water content increased from 0.02 to 0.78% in the presence of an equal amount of the R-mandelate salt of Compound 1 (forward slash bars), while the solubility profile of the corresponding hemihydrate form did not show a similar dependence on water content (backward slash bars), as illustrated in FIG. 8. It appears that the solubility of the hemihydrate is approximately constant within the water test range (with some small random variability in the range of ±1% observed). It also should be noted that the solubility difference between the anhydrous and hemihydrate R-mandelate salts of Compound 3 is minimized in the presence of the R-mandelate salt of Compound 1 with the water content at 0.78% in acetonitrile.

The solubility data indicates that increasing water level during the production of the R-mandelate salt of Compound 1 to 0.7% significantly improves the solubility of the anhydrous form with very little impact on the solubility of the hemihydrate form. Thus, by increasing the process stream water content to 0.7% one can effectively suppress the crystallization of the undesired R-mandelate salt of Compound 3 and significantly improve the robustness of the resolution step during routine manufacturing.

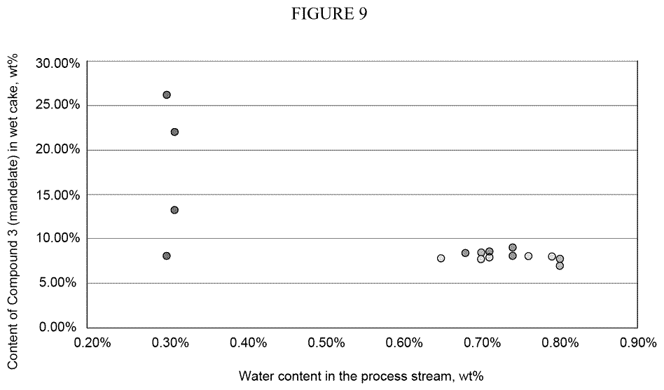

In FIG. 9, the relationship between the water content of the process stream (before crystallization) and the chiral impurity level in the wet cake (after crystallization) has been summarized for past manufacturing campaigns. As shown, when the water content was around 0.3%, there were two batches that ended up with concentrations of the off-isomer content in the isolated wet cake off track. When the water content increased to around 0.7% for the later batches, the off-isomer content stayed at 7%-10%.

### Example 4. Analytical Methods

Details for practicing the various analytical methods described in this document are provided in Table 9-11.

TABLE 9
Assay and Impurity Method Parameters

| Method Parameters | Description |
|---|---|
| Principle: | Reverse phase gradient HPLC method utilizing a liquid chromatograph |
| | system equipped with a UV detector (dual wavelength or PDA) |
| Column: | XSelect, CSH C18, 4.6 × 150 mm, 3.5 μm |
| Mobile Phases | Mobile Phase A: Aqueous 50 mM Sodium Perchlorate, pH 3 |
| | Mobile Phase B: Methanol: Acetonitrile 90:10 |
| Flow Rate | 1.0 mL/min |
| Column | 40° C. |
| Temperature | |
| Gradient | 0-16 min: 90% Mobile Phase A/10% Mobile Phase B |
| | 16-26 min: 10% Mobile Phase A/90% Mobile Phase B |
| | 29 min: 10% Mobile Phase A/90% Mobile Phase B |
| | 29.1 min: 90% Mobile Phase A/10% Mobile Phase B |
| | 44 min: 90% Mobile Phase A/10% Mobile Phase B |
| Detection | 234 nm for Assay and Impurities (excluding Compound 2) |
| | 265 nm for detection of Compound 2 |
| Injection Volume | 10 μL |
| Diluent | Mobile Phase A |
| Standard Solution | 0.6 mg/mL of ulotaront HCl reference standard (0.5 mg/mL ulotaront |
| | free base) in diluent. Check Standard is also prepared at the same |
| | nominal concentration. |
| Sensitivity | 0.0003 mg/mL of ulotaront HCl reference standard (0.00025 mg/mL |
| Verification | ulotaront free base) in diluent. |
| Solution (0.05%) | |
| Retention Time | 0.6 mg/mL of ulotaront HCl reference standard (0.5 mg/mL ulotaront |
| Marker Solution | free base) and 0.00025 mg/mL of Compound 5, Compound 4, and |
| | Compound 2 in diluent. |
| System Suitability | Sensitivity: The ratio of ulotaront peak areas between Sensitivity |
| Criteria | Verification Solution and Standard Solution at 234 nm is within 20% of |
| | the ratio of concentrations (theoretical ratio) |
| | The USP signal-to-noise ratio for Compound 2 peak at 265 nm in the |
| | Retention Time Marker Solution is not less than (NLT) 10. |
| | Injection Precision Acceptance Criteria: The relative standard deviation |
| | of ulotaront peak areas in five consecutive injections of Standard |
| | Solution should be not more than (NMT) 0.7% |
| | Standard Check Acceptance Criteria: The ratio of areas of ulotaront |
| | peak between the standard solution and standard check solution is |
| | within 1.0% of the ratio of their concentrations (theoretical ratio) |
| | Bracketing Standard Acceptance Criteria: The ratio of areas of ulotaront |
| | peak between the standard solution injections and bracketing standard |
| | injections is within 1.0% of the ratio of their concentrations (theoretical |
| | ratio) |
| | Criteria for Retention Time Marker Solution |
| | USP Resolution between ulotaront and Compound 5 is NLT 4.0 |
| | USP Resolution between Compound 5 and Compound 4 is NLT 1.2 |
| Sample Preparation | 0.6 mg/mL ulotaront HCl (0.5 mg/mL ulotaront free base) sample in |
| (in duplicate) | diluent. |
| Reporting | 0.05% |
| Threshold | |
| Calculations: Assay | $\% Assay = \frac{{A}_{sam} \times {C}_{std} \times {V}_{sam} \times 100}{{A}_{std} \times {W}_{sam}}$ |
| | Where: |
| | Aₛₐₘ = Ulotaront peak area in sample injection |
| | A_{std} = Average ulotaront peak area in standard injections |
| | C_{std} = Concentration of ulotaront freebase in working standard |
| | solution with purity correction, in mg/mL |
| | Vₛₐₘ = Volume of sample solution |
| | Wₛₐₘ = Weight of the sample |
| Calculations: Impurities | $\% impurity = \frac{{A}_{sam} \times {C}_{std} \times {V}_{sam} \times 100}{{A}_{std} \times RRF \times {W}_{sam}}$ |
| | Where: |
| | Aₛₐₘ = Impurity peak area in sample injection |
| | A_{std} = Average ulotaront peak area in standard injections at 234 nm |
| | C_{std} = Concentration of ulotaront freebase in working standard |
| | solution with purity correction, in mg/mL |
| | Vₛₐₘ = Volume of sample solution |
| | Wₛₐₘ = Weight of the sample |
| | RRF = Relative Response Factor (RRF for Compound 5 = 0.76, RRF |
| | for Compound 2 = 1.9) |

TABLE 10
Chiral Purity and Identification Method Parameters

| Method Parameter | Description |
|---|---|
| Principle: | Normal phase isocratic HPLC method |
| Column | Chiralcel OD 4.6 × 250 mm, 10 μm (Chiral Technologies) |
| Mobile phase: | Hexanes/Ethanol/EtSO3 (85:15:0.05) |
| Flow Rate | 1 mL/min |
| Injection Volume | 10 μL |
| Detection | 235 nm |
| Wavelength | |
| Diluent | 50:50 mobile phase: ethanol |
| Method Parameter | Description |
| Retention Time | Prepare a standard solution in diluent containing 1.0 mg/mL of ulotaront |
| Marker Solution: | HCI reference standard and 0.02 mg/mL racemic ulotaront HCl |
| | reference material. |
| Sensitivity | 0.001 mg/mL of racemic ulotaront HCI (0.0005 mg/mL ulotaront HCl) |
| Verification | |
| Solution (0.05%) | |
| System Suitability | Sensitivity: The ratio of ulotaront peak areas between Sensitivity |
| Criteria | Verification Solution and Standard Solution is between 0.02% and |
| | 0.08% |
| | USP Resolution between ulotaront and Compound 3 peaks in Retention |
| | Timer Marker solution is NLT 1.5 |
| Sample Solution: | 1.0 mg/mL of ulotaront HCl sample |
| Calculation: | Calculate the amount of Compound 3 using the peak area percentage |
| | method. |
| Chiral | Identification is based on the retention time of ulotaront (+1.0 minute) |
| identification | and the ulotaront peak area agreement. The ulotaront peak area of the |
| | Sample Solution must be within 30% of the peak area of the Standard |
| | Solution |

TABLE 11
Particle Size Distribution Method

| Parameter | Description |
|---|---|
| Model: | Malvern Mastersizer 3000 |
| Sample dispersion unit: | Hydro MV |
| Stirrer Speed: | 2500 rpm |
| Particle shape: | Non-spherical (Mie approximation) |
| Particle type: | RI = 1.5, A I = 1 |
| Dispersant refractive index | 1.38 |
| Sample measurement time | 15 s |
| Background measurement time | 15 s |
| Number of sample preparations | 3 |
| Pre-measurement delay | 60 s |
| Result unit | Volume |
| Obscuration limit | 5-20% |
| Calculation | Automatically calculated by software |
| | algorithm |
| Dispersant solution | 0.2% Span85 in Hexane |
| Standard preparation | Not applicable. Periodic calibration |
| | performed with NIST traceable standards |
| Sample preparation | Add ulotaront HCl sample using a spatula to |
| | 120 mL of dispersant. Measure 3 sample |
| | preparations. |

Throughout this application, various publications are referenced. The disclosures of these publications in their entireties are hereby incorporated by reference into this application in order to more fully describe the state of the art to which this disclosure pertains. It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1-16. (canceled)

17. Ulotaront HCl particles having a D50 of from 37 to 98 μm.

18. The particles of claim 17 having a D10≥8 μm and a D90≤252 μm.

19. The particles of claim 17 in an isolated state.

20. The particles of claim 17, further comprising:
a) Compound 2 in an amount of ≤0.15%, 0.08%, or 0.05% w/w, preferably ≥ 0.001%;
b) Compound 3 in an amount of ≤0.30%, 0.10%, or 0.05% w/w, preferably ≥0.001%;
c) Compound 4 in an amount of ≤0.15%, 0.08%, or 0.05% w/w, preferably ≥ 0.001%; or
d) Compound 5 in an amount of ≤0.60%, 0.30%, 0.10%, or 0.05% w/w, preferably ≥0.001%;
wherein the percentages are based on the weight of the free base of ulotaront and the free base of the compound.

21. The particles of claim 17, wherein the ulotaront HCl comprises from 0.01% to 0.5% w/w water or from 0.02% to 0.09% w/w water.

22. A composition comprising ulotaront HCl and a compound selected from:
a) Compound 2 in an amount of ≤0.15%, 0.08%, or 0.05% w/w, preferably ≥ 0.001%;
b) Compound 3 in an amount of ≤0.30%, 0.10%, or 0.05% w/w, preferably ≥ 0.001%;
c) Compound 4 in an amount of ≤0.15%, 0.08%, or 0.05% w/w, preferably ≥0.001%; or
d) Compound 5 in an amount of ≤0.60%, 0.30%, 0.10%, or 0.05% w/w, preferably ≥0.001%;
wherein the percentages are based on the weight of the free base of ulotaront and the free base of the compound.

23. The composition of claim 22 in an isolated state.

24. The composition of claim 22, wherein the ulotaront HCl comprises from 0.01% to 0.5% w/w water or from 0.02% to 0.09% w/w water.

25. A solid drug formulation comprising granulates comprising 30 to 120 mg of ulotaront HCl particles in combination with one or more intra-granular excipients selected from a disintegrant, a diluent, a lubricant, or a combination thereof, further comprising a compound selected from:
a) Compound 2 in an amount of ≤0.15%, 0.08%, or 0.05% w/w, preferably ≥ 0.001%;
b) Compound 3 in an amount of ≤0.30%, 0.10%, or 0.05% w/w, preferably ≥ 0.001%;
c) Compound 4 in an amount of ≤0.15%, 0.08%, or 0.05% w/w, preferably ≥0.001%; or
d) Compound 5 in an amount of ≤0.60%, 0.30%, 0.10%, or 0.05% w/w, preferably ≥0.001%;
wherein the percentages are based on the weight of the free base of ulotaront and the free base of the compound.

26. The formulation of claim 25, wherein the ulotaront HCl comprises from 0.01% to 0.5% w/w water, or from 0.02% to 0.09% w/w water.

27. (canceled)

28. Ulotaront HCl comprising from 0.01% to 0.5% w/w water.

29. (canceled)

30. (canceled)

31. A solid drug formulation comprising 30 to 120 mg of the ulotaront HCl claim 28 in combination with one or more excipients selected from a disintegrant, a diluent, a lubricant, or a combination thereof.

32. The formulation of claim 31 comprising the ulotaront HCl as granulates wherein the ulotaront HCl comprises from 0.01% to 0.5% w/w water in combination with one or more intra-granular excipients selected from a disintegrant, a diluent, a lubricant, or a combination thereof.

33. The formulation of claim 31, wherein the ulotaront HCl comprises from 0.02% to 0.09% w/w water.

34. A method of making a pharmaceutical dosage form comprising:
a) providing a lot of ulotaront HCl comprising an amount of impurity, wherein the impurity is selected from Compound 2, Compound 3, Compound 4, and Compound 5, or a salt thereof;
b) chromatographically separating the impurity from the ulotaront in a sample from the lot and determining the amount of impurity in the sample; and
c) if the amount of the impurity in the sample is less than or equal to an acceptance threshold, processing the lot into one or more finished dosage forms.

35-60. (canceled)

61. A solid drug formulation comprising granulates comprising 30 to 120 mg of the ulotaront HCl particles of claim 17, in combination with one or more intra-granular excipients selected from a disintegrant, a diluent, a lubricant, or a combination thereof.

62. The formulation of claim 61, wherein the ulotaront HCl particles have a D10≥8 μm and a D90≤252 μm.

63. The formulation of claim 61, wherein the intra-granular excipients pass through a 30 mesh sieve; and/or wherein the lubricant passes through a 60 mesh sieve.

64. The formulation of claim 61, comprising from 20 to 50% w/w or from 25 to 45% w/w ulotaront HC1, wherein the % w/w is based on the weight of the formulation.

65. The formulation of claim 61, in the form of a tablet compressed from a mixture having one or more characteristics selected from:
a) a critical orifice diameter (“COD”) of from 9 to 26 mm,
b) a bulk density of from 0.42 to 0.48 g/cc,
c) a tap density (250 taps) of from 0.51-0.60 g/cc,
d) a tap density (500 taps) of from 0.52-0.61 g/cc,
e) a Carr's Index (CI) of from 19.0 to 27.1, and
f) a Hausner Ratio (“HR”) of from 1.23 to 1.37.

66. The formulation of claim 61, in the form of a tablet compressed at a compression pressure of from 140 to 400 MPa, 160 to 360 MPa, 150 to 300 MPa, from 170 to 250 MPa, or from 180 to 220 MPa, and/or having a tensile strength of from 0.8 to 4.5 MPa, 1.5 to 4.0 MPa, or 1.9 to 3.3 MPa.

67. The formulation of claim 61, further comprising a compound selected from:
a) Compound 2 or a salt thereof in an amount of ≤0.15%, 0.08%, or 0.05% w/w, preferably ≥0.001%;
b) Compound 3 or a salt thereof in an amount of ≤0.30%, 0.10%, or 0.05% w/w, preferably ≥0.001%;
c) Compound 4 or a salt thereof in an amount of ≤0.15%, 0.08%, or 0.05% w/w, preferably ≥0.001%; or
d) Compound 5 or a salt thereof in an amount of ≤0.60%, 0.30%, 0.10%, or 0.05% w/w, preferably ≥0.001% w/w;
wherein the percentages are based on the weight of the free base of ulotaront and the free base of the compound.

68. The formulation of claim 61, further comprising a compound selected from:
a) Compound 2 or a salt thereof preferably in an amount of ≤0.15% w/w;
b) Compound 3 or a salt thereof preferably in an amount of ≤0.30% w/w;
c) Compound 4 or a salt thereof preferably in an amount of ≤0.15% w/w; or
d) Compound 5 or a salt thereof preferably in an amount of ≤0.60% w/w;
wherein the percentages are based on the weight of the free base of ulotaront and the free base of the compound.

69. The formulation of claim 61, wherein the ulotaront HCl comprises from 0.01% to 0.5% w/w water, or from 0.015% to 0.2% w/w water, or from 0.02% to 0.09% w/w water.
